SEQUENCE LISTING (4 sequences)

SEQ ID NO: 1 - protein, 106 aa, Artificial Sequence
  Description of Artificial Sequence Synthetic polypeptide
QIQLTQSPSSLSASVGDRVTITCSASSSVNHMFWYQQKPGKAPKPWIYLTSNLASGVPSRFSGSGSGTDYTLTISSLQPEDFATYYCQQWSGNPWTFGQGTKVEIK

SEQ ID NO: 2 - protein, 118 aa, Artificial Sequence
  Description of Artificial Sequence Synthetic polypeptide
EVQLVESGGGLVQPGGSLRLSCAASGFTFSRYTMSWVRQAPGKGLEWVATISGGGHTYYLDSVKGRFTISRDNSKNTLYLQMNSLRAEDTAVYYCTRGFGDGGYFDVWGQGTLVTVSS

SEQ ID NO: 3 - protein, 213 aa, Artificial Sequence
  Description of Artificial Sequence Synthetic polypeptide
QIQLTQSPSSLSASVGDRVTITCSASSSVNHMFWYQQKPGKAPKPWIYLTSNLASGVPSRFSGSGSGTDYTLTISSLQPEDFATYYCQQWSGNPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 447 aa, Artificial Sequence
  Description of Artificial Sequence Synthetic polypeptide
EVQLVESGGGLVQPGGSLRLSCAASGFTFSRYTMSWVRQAPGKGLEWVATISGGGHTYYLDSVKGRFTISRDNSKNTLYLQMNSLRAEDTAVYYCTRGFGDGGYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

(19) United States
(12) Patent Application Publication
(10) Pub. No.: US 2016/0340433 A1
(43) Pub. Date: Nov. 24, 2016

(54) SELECTION AND TREATMENT OF SUBJECTS

(71) Applicant: Santarus, Inc., Bridgewater, NJ (US)

(72) Inventor: Mark Totoritis, Rancho Santa Fe, CA (US)

(21) Appl. No.: 15/228,711

(22) Filed: Aug. 4, 2016

Related U.S. Application Data
(63) Continuation of application No. 13/981,699, filed on Oct. 3, 2013.
(60) Provisional application No. 61/439,348, filed on Feb. 3, 2011.
(60) Provisional application No. 61/498,263, filed on Jun. 17, 2011.

(51) Int. Cl. C07K 16/28; A61K 31/655; A61K 31/4706; A61K 31/519; A61K 39/395; A61K 31/42

(52) U.S. Cl. CPC C07K 16/2842; A61K 39/3955; A61K 31/42; A61K 31/4706; A61K 31/519; A61K 31/655; C07K 2317/56; A61K 2039/505

(57) ABSTRACT

The invention relates to methods of selecting a subject, and methods of treating the subject with an anti-VLA-1 antibody. In one embodiment the first therapeutic agent is a DMARD (Disease Modifying Antirheumatic Drug), such as gold salts; hydroxychloroquine; an antifolate, such as methotrexate; a pynmidine synthesis inhibitor, such as leflunomide; or a sulfa drug, such as sulfasalazine. For example, the DMARD can be methotrexate, administered at a dose of mg/week or less; leflunomide, administered at a dose of 20 mg/day or less; sulfasalazine, administered at a dose of 3000 mg/day or less; or hydroxychloroquine, administered at a dose of 400 mg/day or less.

### RELATED APPLICATIONS

This application claims the benefit of U.S. Provisional Application No. 61/439,348, filed Feb. 3, 2011, and U.S. Provisional Application No. 61/498,263, filed Jun. 17, 2011. Both prior applications are incorporated herein by reference in their entirety.

### FIELD OF THE INVENTION

The invention relates to methods of selecting a subject, and methods of treating the subject with an anti-VLA-1 antibody.

### BACKGROUND OF INVENTION

Integrins are a superfamily of cell surface receptors that mediate cell-cell and cell-matrix adhesion. These heterodimeric proteins, composed of two noncovalently linked polypeptide chains, α and β, provide anchorage as well as signals for cellular growth, migration and differentiation during development and tissue repair. Integrins have also been implicated in immune and inflammatory processes, which require the extravasation of cells out of blood vessels, into tissues and towards the site of infection.

VLA-1 (also called α1β1) belongs to a class of integrins called VLA (“Very Late Antigen”) integrins. VLA-1 binds collagen (both types I and IV) and laminin, and has been implicated in cell adhesion and migration on collagen; contraction and reorganization of collagen matrices; and regulation of expression of genes involved in extracellular matrix remodeling.

VLA-1 has been shown to be involved in the development of rheumatoid arthritis, a chronic inflammatory disease associated with bone resorption. Infiltrating T cells in the arthritic synovium of patients express high levels of VLA-1, and its blockade with antibodies significantly reduces the inflammatory response and the development of arthritis in animal models.

SUMMARY OF INVENTION

The invention is based, at least in part, on the discovery of new and improved methods of treating a subject with an anti-VLA-1 antibody. In one aspect, the invention features a method wherein a subject, such as a patient who has an inflammatory disorder, such as arthritis, is selected as a candidate to receive treatment with an anti-VLA-1 antibody if the patient has previously received treatment with at least one first therapeutic agent, and optionally, the response of the patient to the first therapeutic agent failed to meet a predetermined criterion. For example, the patient may have failed to experience relief of arthritic symptoms after a given amount of time, such as over the course of two weeks or one month or two months, or longer. If the response of the patient to the first therapeutic agent does meet a predetermined criterion or response level, then the patient is typically not selected to receive treatment with an anti-VLA-1 antibody.

In one embodiment, the patient fails to meet a predetermined criterion when (i) the patient fails to have an improvement in arthritic symptoms; (ii) the patient ceases to have improvement in arthritic symptoms; or (iii) the patient experiences a worsening of arthritic symptoms. An improvement in arthritic symptoms can be manifested for example by a decrease in swollen joint count or tender joint count. A worsening of arthritic symptoms can be manifested by an increase in swollen joint count or tender joint count. An improvement or worsening of symptoms can also be assayed by the amount of pain reported by the patient following administration of the first agent, by the amount of RF (rheumatoid factor) identified in the blood of the patient, or the quality of the joint synovial fluid collected from the patient. For example, an improvement of symptoms can be indicated by a decrease in the number of white blood cells (WBCs) or peripheral blood mononucleocytes (PMNs) in the joint synovial fluid.

A subject, for example, a patient identified as a candidate to receive treatment with an anti-VLA-1 antibody by a method described herein, can be administered the anti-VLA-1 antibody. In one embodiment, the patient has arthritis, such as rheumatoid arthritis, and the patient received a diagnosis of having the arthritis at least six months before being selected to receive treatment with an anti-VLA-1 antibody. In another embodiment, the patient has an inflammatory bowel disease (IBD), such as ulcerative colitis or Crohn's Disease, and the patient received a diagnosis of having the IBD at least six months before being selected to receive treatment with an anti-VLA-1 antibody.

In one embodiment the first therapeutic agent is a DMARD (Disease Modifying Antirheumatic Drug), such as gold salts; hydroxychloroquine; an antifolate, such as methotrexate; a pyrimidine synthesis inhibitor, such as leflunomide; or a sulfa drug, such as sulfasalazine. For example, the DMARD can be methotrexate, administered at a dose of 25 mg/week or less; leflunomide, administered at a dose of 20 mg/day or less; sulfasalazine, administered at a dose of 3000 mg/day or less; or hydroxychloroquine, administered at a dose of 400 mg/day or less.

In another embodiment, the first therapeutic agent is a TNF-α inhibitor, such as an anti-TNF-α antibody, such as, for example, infliximab, adalimumab, certolizumab pegol, or golimumab; or the fusion protein etanercept.

In another embodiment, the first therapeutic agent is an inhibitor of VLA-2, such as an anti-VLA-2 antibody, for example GBR 500.

In yet another embodiment, the first therapeutic agent is an inhibitor of an integrin, such as MAdCAM-1 (Mucosal Vascular Addressin Cell Adhesion Molecule-1, α4β7 integrin). The MAdCAM-1 inhibitor can be an anti-MAdCAM-1 antibody, such as vedolizumab (MLN0002, Millennium Pharmaceuticals, Cambridge, Mass.). For example, in one embodiment, the patient has an inflammatory bowel disease, and the patient had an inadequate response to treatment with an anti-MAdCAM-1 antibody prior to receiving treatment with an anti-VLA-1 antibody.

In another embodiment, the first therapeutic agent is a B cell-depleting agent, such as an anti-CD20 antibody, for example rituximab (Rituxan, Genentech, Inc., South San Francisco, Calif.; and IDEC Pharmaceutical, San Diego, Calif.).

In another embodiment, the first therapeutic agent is an inhibitor of a Janus kinase

(JAK) family member or a Spleen tyrosine kinase (SYK) family member. JAK family members include JAKE JAK2, JAK3 and TYK2, and SYK family members include SYK and ZAP-70. In one embodiment, the first therapeutic agent is an inhibitor of JAK3, such as the small molecule inhibitor CP-690,550 (tofacitinib). In another embodiment, the first therapeutic agent is a SYK inhibitor, such as the small molecule inhibitor R406, or its prodrug R788.

In one embodiment, administration of the first therapeutic agent is stopped before administration of the anti-VLA-1 antibody. For example, administration of the first therapeutic agent can be stopped at least 2 weeks, at least 3 weeks, at least 4 weeks, at least 5 weeks or more before administration of the anti-VLA-1 antibody. In one embodiment, the patient will not be administered an anti-VLA-1 antibody before the patient has cleared a therapeutic amount of the first therapeutic agent from the body. Similarly, the patient may not be administered the first therapeutic agent while the patient has therapeutic levels of anti-VLA-1 antibodies in the body.

In some embodiments, the patient continues to receive the first therapeutic agent when the anti-VLA-1 antibody is administered. For example, the patient may continue to receive a DMARD, or more than one DMARD, when the anti-VLA-1 antibody is administered. In other embodiments, the patient will not receive more than one DMARD while receiving treatment with the anti-VLA-1 antibody. In one embodiment, the patient receives treatment with a DMARD and hydroxychloroquine while receiving treatment with an anti-VLA-1 antibody.

In one embodiment, the patient receives an administration of the first therapeutic agent after an administration of the anti-VLA-1 antibody therapy, or the administrations are selected such that therapeutic levels of both the antibody and the first therapeutic agent are maintained in the patient. For example, the antibody and the first therapeutic agent can be maintained in the body for at least 1 day, at least 2 days, at least 5 days, or at least 10 days or more.

In one embodiment, the patient continues to receive treatment with the first therapeutic agent, which is, for example, methotrexate, leflunomide, sulfasalazine or hydroxychloroquine, while the patient is also administered the anti-VLA-1 antibody. For example, in one embodiment, the first therapeutic agent is methotrexate, and the methotrexate is administered at a dose of 35 mg/week, 30 mg/week, 25 mg/week, 20 mg/week, or 15 mg/week, or less while the patient is also administered the anti-VLA-1 antibody. In another embodiment, the first therapeutic agent is leflunomide, and the leflunomide is administered at a dose of 30 mg/day, 25 mg/day, 20 mg/day, 15 mg/day, 10 mg/day, or less while the patient is also administered the anti-VLA-1 antibody. In another embodiment, the first therapeutic agent is sulfasalazine, and the sulfasalazine is administered at a dose of 4000 mg/day, 3500 mg/day, 3000 mg/day, 2500 mg/day, 2000 mg/day or less while the patient is also administered the anti-VLA-1 antibody. In another embodiment, the first therapeutic agent is hydroxychloroquine, and the hydroxychloroquine is administered at a dose of 500 mg/day, 450 mg/day, 400 mg/day, 350 mg/day, 300 mg/day, or less while the patient is also administered the anti-VLA-1 antibody.

In yet another embodiment, the first therapeutic agent is hydroxychloroquine, and the patient is further administered a second DMARD while the patient is also administered the anti-VLA-1 antibody.

In one embodiment, the patient continues to receive treatment with the first therapeutic agent, which is an anti-MAdCAM-1 antibody, such as vedolizumab, while the patient is also administered the anti-VLA-1 antibody. For example, in one embodiment, the anti-MAdCAM-1 antibody is administered at a dose of 20 mg/kg, 15 mg/kg, 10 mg/kg, 6 mg/kg, 2 mg/kg or less every two weeks by a suitable route of administration, such as by intravenous (IV) injection, while the patient is also administered the anti-VLA-1 antibody.

In one embodiment, the anti-VLA-1 antibody includes a light chain polypeptide comprising the sequence of SEQ ID NO:1, and a heavy chain polypeptide comprising the sequence of SEQ ID NO:2. For example, the anti-VLA-1 antibody can include a light chain polypeptide comprising the sequence of SEQ ID NO:3, and a heavy chain polypeptide comprising the sequence of SEQ ID NO:4.

In one embodiment, the anti-VLA-1 antibody binds the same epitope as an antibody having a light chain polypeptide comprising the sequence of SEQ ID NO:1, and a heavy chain polypeptide comprising the sequence of SEQ ID NO:2.

In one embodiment, a method of treating a patient with an anti-VLA-1 antibody is provided, where the patient was previously administered a first therapeutic agent, and where the response to the first therapeutic agent was assessed and determined to be inadequate. The method includes administering an effective amount of an anti-VLA-1 antibody to the patient. The response can be determined to be inadequate if, for example, (i) the patient failed to have an improvement in symptoms; (ii) the patient ceased to have improvement in symptoms; or (iii) the patient experienced a worsening of symptoms.

For example, in one embodiment, the patient has arthritis, such as rheumatoid arthritis, and the response is determined to be inadequate if (i) the patient failed to have an improvement in arthritic symptoms; (ii) the patient ceased to have improvement in arthritic symptoms; or (iii) the patient experienced a worsening of arthritic symptoms.

An improvement in arthritic symptoms can be manifested by a decrease in swollen joint count or tender joint count, and a worsening of arthritic symptoms can be manifested by an increase in swollen joint count or tender joint count.In one embodiment, the patient has an IBD, such as ulcerative colitis or Crohn's Disease, and the response is determined to be inadequate if (i) the patient failed to have an improvement in IBD symptoms; (ii) the patient ceased to have improvement in IBD symptoms; or (iii) the patient experienced a worsening of IBD symptoms.

In one embodiment the first therapeutic agent is a DMARD, such as gold salts; hydroxychloroquine; an antifolate, such as methotrexate; a pyrimidine synthesis inhibitor, such as leflunomide; or a sulfa drug, such as sulfasalazine. In other embodiments, the first therapeutic agent is a TNF-α inhibitor, a JAK inhibitor or a SYK inhibitor, an anti-VLA-2 antibody, such as GBR 500; an anti-MAdCAM-1 antibody, such as vedolizumab; or an anti-CD20 antibody, such as rituximab.

In one embodiment, the invention features a method of treating a patient with an anti-VLA-1 antibody, where the patient was previously administered a first therapeutic agent, and where the response to the first therapeutic agent was inadequate. The method includes administering an effective amount of an anti-VLA-1 antibody to the patient.

In one embodiment, a method of treating a patient with an anti-VLA-1 antibody is provided, where the patient was previously administered a first therapeutic agent, and responsive to a negative assessment of the patient's response to the first therapeutic agent, such as an assessment that the response failed to meet a predetermined criterion, the patient is administered an effective amount of an anti-VLA-1 antibody to the patient. The negative assessment can be acquired directly or indirectly.

In one embodiment, a method of treating a patient is provided, which comprises administering a first therapeutic agent, which is an anti-VLA-1 antibody, and a second therapeutic agent, where administering the first and the second therapeutic agents is effective to treat arthritis in the patient. The second therapeutic agent can be, for example, a DMARD, a TNF-α inhibitor, a JAK inhibitor (for example, an inhibitor of JAK1, JAK2, JAK3 or TYK2), a SYK inhibitor (for example, an inhibitor of SYK or ZAP-70), a VLA-2 inhibitor, an IL-6 inhibitor, an IL-17 inhibitor, an IL-12/IL-23 inhibitor, a MAdCAM-1 inhibitor, a CD20 inhibitor or another biologic agent. For example, the second therapeutic agent can be methotrexate, leflunomide, sulfasalazine, or hydroxychloroquine, GBR 500, infliximab, adalimumab, certolizumab pegol, golimumab, etanercept, rituximab, tocilizumab, abatacept, or vedolizumab.

In one embodiment, the second therapeutic agent is methotrexate, administered at a dose of 35 mg/week, 30 mg/week, 25 mg/week, 20 mg/week, or 15 mg/week, or less. In another embodiment, the second therapeutic agent is leflunomide, administered at a dose of 30 mg/day, 25 mg/day, 20 mg/day, 15 mg/day, 10 mg/day or less. In another embodiment the second therapeutic agent is sulfasalazine, administered at a dose of 4000 mg/day, 3500 mg/day, 3000 mg/day, 2500 mg/day, 2000 mg/day, or less. In another embodiment, the second therapeutic agent is hydroxychloroquine, administered at a dose of 500 mg/day, 450 mg/day, 400 mg/day, 350 mg/day, 300 mg/day or less.

In another embodiment, the second therapeutic agent is an antibody, such as an anti-MAdCAM-1 antibody, such as vedolizumab, and the antibody is administered at a dose of, for example, 20 mg/kg, 15 mg/kg, 10 mg/kg, 6 mg/kg, 2 mg/kg or less every two weeks by a suitable route of administration, such as by intravenous (IV) injection.

In one embodiment, the patient is administered a third therapeutic agent, which can be, for example, a DMARD, such as gold salts; hydroxychloroquine; an antifolate, such as methotrexate; a pyrimidine synthesis inhibitor, such as leflunomide; or a sulfa drug, such as sulfas alazine; a TNF-α inhibitor, such as an anti-TNF-α antibody, such as, for example, infliximab, adalimumab, certolizumab pegol, or golimumab; or the fusion protein etanercept; a VLA-2 inhibitor, such as an anti-VLA-2 antibody, such as GBR 500; a MAdCAM-1 inhibitor, such as an anti-MAdCAM-1 antibody, such as vedolizumab; a B cell-depleting agent, such as a CD20 inhibitor, such as an anti-CD20 antibody, for example rituximab; a JAK inhibitor, such as tofacitinib; or a SYK inhibitor, such as R406, or the prodrug R788. In one embodiment, the patient has an IBD, such as ulcerative colitis or Crohn's Disease, and the second therapeutic agent or the third therapeutic agent is a MAdCAM-1 inhibitor, such as an anti-MAdCAM-1 antibody, such as vedolizumab.

In one embodiment, administration of the first and second, and optionally the third, therapeutic agents results in a greater improvement of symptoms than is observed following administration of either the first or the second (or third) therapeutic agents alone.

In one embodiment, a method of selecting a patient as a candidate to receive treatment with an anti-VLA-1 antibody is provided, where the patient previously has been administered a first therapeutic agent. The method includes performing a test on a patient sample to assess a patient's response to the first therapeutic agent, and if the patient response to the first therapeutic agent fails to meet a predetermined criterion, selecting the patient as a candidate for treatment with an anti-VLA-1 antibody. If the patient's response to the first therapeutic agent does meet the predetermined criterion, the patient is determined not to be a candidate to receive treatment with the anti-VLA-1 antibody. The patient may have arthritis, for example, rheumatoid arthritis.

The patient may be selected as a candidate for treatment with an anti-VLA-1 antibody, if (i) the patient fails to have an improvement in arthritic symptoms; (ii) the patient ceases to have improvement in arthritic symptoms; or (iii) the patient experiences a worsening of arthritic symptoms.

An improvement in arthritic symptoms can be manifested by a decrease in swollen joint count or tender joint count, and a worsening of arthritic symptoms can be manifested by an increase in swollen joint count or tender joint count.

In one embodiment, an effective amount of an anti-VLA-1 antibody is administered to the patient who is selected as a candidate for treatment with the antibody.

In one aspect, the invention features a method of selecting or classifying a patient as a candidate to receive treatment with an anti-VLA-1 antibody, where the patient previously has been administered a first therapeutic agent. The method includes assessing a patient's response to the first therapeutic agent, and if the response fails to meet a predetermined criterion, selecting or classifying the patient as a candidate for treatment with an anti-VLA 1 antibody. If the response does meet a predetermined criterion, the patient is selected or classified as not being a candidate to receive treatment with the anti-VLA-1 antibody. Assessing the patient's response can include analyzing a sample, such as a tissue or joint fluid sample, from the patient.

In another embodiment, a method of treating a patient by administering a first therapeutic agent to the patient is provided, where the first therapeutic agent is an anti-VLA-1 antibody, and further administering a second therapeutic agent to the patient, where the second therapeutic agent is an anti-inflammatory agent. Administration of the first and second therapeutic agents is effective to treat an inflammatory disease, such as arthritis, for example, rheumatoid arthritis, in the patient.

In one embodiment the second therapeutic agent is methotrexate, leflunomide, sulfasalazine, or hydroxychloroquine. For example, the second therapeutic agent can be methotrexate, administered at a dose of, for example, 35 mg/week, 30 mg/week, 25 mg/week, 20 mg/week, or 15 mg/week, or less; the second therapeutic agent can be leflunomide, administered at a dose of, for example, 30 mg/day, 25 mg/day, 20 mg/day, 15 mg/day, 10 mg/day or less; the second therapeutic agent can be sulfasalazine, administered at a dose of, for example, 4000 mg/day, 3500 mg/day, 3000 mg/day, 2500 mg/day, 2000 mg/day, or less; or the second therapeutic agent can be hydroxychloroquine, administered at a dose of, for example, 500 mg/day, 450 mg/day, 400 mg/day, 350 mg/day, 300 mg/day, or less.

In another embodiment, the patient is administered a third therapeutic agent, such as a DMARD, such as gold salts; hydroxychloroquine; an antifolate, such as methotrexate; a pyrimidine synthesis inhibitor, such as leflunomide; or a sulfa drug, such as sulfasalazine; a TNF-α inhibitor, such as an anti-TNF-α antibody, such as, for example, infliximab, adalimumab, certolizumab pegol, or golimumab; or the fusion protein etanercept; a VLA-2 inhibitor, such as an anti-VLA-2 antibody, such as GBR 500; a MAdCAM-1 inhibitor, such as an anti-MAdCAM-1 antibody, such as vedolizumab; a B cell-depleting agent, such as a CD20 inhibitor, such as an anti-CD20 antibody, for example rituximab; a JAK inhibitor, such as tofacitinib; or a SYK inhibitor, such as R406, or the prodrug R788. Typically, the first, second, and third agents are different from each other.

In one embodiment, the patient has an IBD, such as ulcerative colitis or Crohn's Disease, and the second therapeutic agent or the third therapeutic agent is a MAdCAM-1 inhibitor, such as an anti-MAdCAM-1 antibody, such as vedolizumab.

In one embodiment, administration of the first and second therapeutic agents results in a greater improvement of symptoms, such as symptoms of rheumatoid arthritis or IBD, than when either first or second therapeutic agent is administered alone.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIGS. 1A and 1B arc a light chain variable domain sequence (SEQ ID NO:1) and a heavy chain variable domain sequence (SEQ ID NO:2), respectively, for an anti-VLA-1 antibody. These sequences include the light chain and heavy chain CDRs, respectively.

FIGS. 2A and 2B are the sequences of a light chain polypeptide (SEQ ID NO:3) and a heavy chain polypeptide (SEQ ID NO:4), respectively, for an anti-VLA-1 antibody.

### DETAILED DESCRIPTION

The invention is based, at least in part, on the discovery of new and improved methods of treating a patient with an anti-VLA-1 antibody. Accordingly, in one method, a patient who has received a first therapy for a certain amount of time is switched to a different therapy, which includes treatment with an anti-VLA-1 antibody. The patient is selected for treatment with the anti-VLA-1 antibody if, for example, the patient fails to achieve or maintain a preselected level of improvement in response to treatment with a first-line therapy, or stops responding to the first-line therapy. For example, the patient may fail to meet a preselected criterion for improvement, or exhibit an unacceptable level of symptoms, or fail to meet a preselected criterion for symptoms following administration of the first-line therapy. In some cases, the patient will have received more than one therapy before being selected for treatment with the anti VLA-1 antibody. In one embodiment, the patient will have failed to achieve or maintain a preselected level of improvement with more than one prior therapy. For example, the patient may have failed to meet a preselected level of improvement or exhibited an unacceptable level of symptoms, or failed to meet a preselected criterion for symptoms following treatment with more than one prior therapy. In these cases, the patient can be classified as an inadequate responder to the first-line therapy, or to the one or more prior therapies, or can be classified as a patient who has received a negative assessment following administration of the first-line therapy, or to the one or more prior therapies. A patient who has failed to respond to one or more prior therapies can be diagnosed as having refractory disease, such as refractory rheumatoid arthritis.

As used herein, a subject who “fails to achieve” an adequate response means that the subject, during the course of treatment, never demonstrated the preselected level of improvement. As used herein, “ceases to demonstrate,” or “ceases to achieve” or “fails to maintain,” the preselected level of response means that the subject demonstrated, or achieved, the preselected level of response at one time, during the course of treatment, but later, such as days, weeks or months later, experienced a worsening of symptoms, even while continuing to receiving the treatment that led to the initial improvement in symptoms.

Disorders. The methods featured in the invention are particularly suited for the treatment of arthritis, such as autoimmune arthritis, for example, rheumatoid arthritis or psoriatic arthritis; or other forms of inflammatory arthritis, such as arthritis associated with inflammatory bowel disease. The patient selected for treatment with an anti-VLA-1 antibody can have arthritis, for example, rheumatoid arthritis, and can have displayed an inadequate response to a first-line therapy, or to more than one prior therapies, or can have received a negative assessment following administration of the first-line therapy, or to the one or more prior therapies.

Autoimmune arthritis is caused by abnormalities in the immune system that cause the body to start attacking its own joints and connective tissue. Examples of autoimmune arthritis include rheumatoid arthritis, juvenile arthritis, psoriatic arthritis, and ankylosing spondylitis. Rheumatoid arthritis is a chronic syndrome characterized by non-specific, usually symmetric inflammation of the peripheral joints, potentially resulting in progressive destruction of articular and periarticular structures, with or without generalized manifestations. Juvenile arthritis (arthritis beginning at or before age 16) is similar to adult rheumatoid arthritis, and tends to affect large and small joints, and may affect growth and development. Psoriatic arthritis, which occurs in about 7% of psoriasis patients, is an inflammatory arthritis associated with psoriasis of the skin or nails; and a negative test for RF (Rheumatoid factor). Ankylosing spondylitis is a systemic rheumatic disorder characterized by inflammation of the axial skeleton and large peripheral joints.

Other types of arthritis, particularly inflammatory arthritis, are suited for treatment by the methods featured in the invention. For example, arthritis associated with inflammatory bowel disease can be treated with an anti-VLA-1 antibody when a first-line therapy fails or ceases to relieve arthritic symptoms.

Efficacy of an agent for treatment of arthritis may be measured by a number of available diagnostic tools, including but not limited to, for example, physical examination, including assaying the number of tender joint counts or swollen joint counts, joint X-rays, blood tests, or examination of fluid collected from affected joints. X-rays can reveal erosions, cysts and joint space narrowing that can occur in chronic rheumatoid arthritis. Blood tests that indicate elevated ESR (Erythrocyte Sedimentation Rate) levels or the presence of antibodies to altered γ-globulin (i.e., rheumatic factors, “RFs”) are indicative of rheumatoid arthritis. Synovial fluid from joints of patients with rheumatoid arthritis is typically cloudy but sterile with reduced viscosity and usually 3,000 to 50,000 white blood cells (WBCs)/μL.

Symptoms of arthritis, such as rheumatoid arthritis, include joint pain, joint swelling, joint deformities, reduced ability to move a joint, redness of the skin around a joint, stiffness, warmth around a joint, morning stiffness, and effusion (collection of liquid in the joints). Criteria for the diagnosis of rheumatoid arthritis is set forth in Aletaha et al., “2010 Rheumatoid Arthritis Classification Criteria,” Arthritis and Rheumatism 62:2569-2581, 2010, and involves the assessment of the number of large and small joints affected in a subject, the levels of RF (rheumatoid factor) and ACPA (anti-citrullinated protein antibody) in serum, CRP (C-reactive protein) and ESR (erythrocyte sedimentation rate) levels, and whether the subject's symptoms have persisted for at least six weeks, or for less than six weeks. The duration of symptoms is determined by the patient's self-report of the duration of signs and symptoms of synovitis (pain, swelling, and tenderness) of any joint that is clinically involved at the time of assessment. Each of these factors provides a score, and a total score ≧6 (on a scale of 0-10), is indicative of rheumatoid arthritis.

“Large joints” include shoulders, elbows, hips, knees and ankles, and “small joints” include metacarpophalangeal, proximal interphalangeal (PIP), second through fifth metatarsophalangeal (MTP), and thumb interphalangeal (IP) joints, and the wrists.

RF and ACPA levels are usually reported in IU (International Units). Based on the upper limit of normal (ULN) for the respective laboratory test and assay the following definitions can be made: negative=less than or equal to the ULN for the laboratory test and assay; low-level positive=higher than the ULN but ≦3 times the ULN for the laboratory test and assay; high-level positive=>3 times the ULN for the laboratory test and assay.

CRP and ESR levels are scored as normal or abnormal based on the local laboratory standards. If results of at least one of these two tests are abnormal, the patient is scored as having an abnormal acute response.

Patients having arthritis, for example, rheumatoid arthritis, also often have an increased level of VLA-1⁺ cells, such as VLA-1⁺ T cells or monocytes.

The methods featured in the invention are also suited for treating autoimmune disorders, such as inflammatory bowel disease (IBD) (for example, ulcerative colitis or

Crohn's disease). In one embodiment, the patient selected for treatment with an anti-VLA-1 antibody has an IBD and has displayed an inadequate response to a first therapy, or to more than one prior therapies, or has received a negative assessment following administration of the first-line therapy, or to the one or more prior therapies.

Efficacy of an agent for treatment of an IBD may be monitored by a number of parameters, including but not limited to, for example, number of liquid or soft stools per day, abdominal pain, presence of abdominal mass, hematocrit of <0.47 in men and <0.42 in women, deviation from standard weight, anal fissures, fistulae or abscesses; and inflammation of iris or uveitis.

The Crohn's Disease Activity Index provides a quantitative assessment of the severity of disease using symptoms such as those described above (Best et al., “Development of a Crohn's Disease Activity Index. National Cooperative Crohn's Disease Study” Gastroenterology 70:439-444, 1976). A CDAI of 220-400 typically indicates moderate to severe Crohn's disease. A CDAI of greater than 450 typically indicates severe disease. Remission of Crohn's Disease is typically defined as a fall in the CDAI of greater than 150. A response to therapy is typically recognized as a fall in CDAI of greater than 70 points.

The quantitative analysis provided by the CDAI (or other similar activity scales, see D′Haens et al. “A Review of Activity Indices and Efficacy End Points for Clinical Trials of Medical Therapy in Adults with Ulcerative Colitis” Gastroeneterology 132:763-786, 2007) is frequently used in conjunction with the qualitative analysis provided by Inflammatory Bowel Disease Questionnaire (IBDQ), which addresses quality of life for patients with Crohn's Disease (Irvine et al., “Quality of Life: a Valid and Reliable Measure of Therapeutic Efficacy in the Treatment of Inflammatory Bowel Disease.

Canadian Crohn's Relapse Prevention Trial Study group” Gastroenterology 106:287-96, 1994). The IBDQ is a 32-item questionnaire that incorporates elements of social, systemic, and emotional symptoms as well as bowel related symptoms into an activity index. The questionnaire addresses bowel function, emotional function, systemic symptoms and social function, and can be self-administered. The total score on the index ranges from 32 to 224, with higher scores indicating better quality of life. The scores of patients in remission usually ranges from 170 to 190. A response is typically defined as an increase in score of 15 points, 16 points, 17 points, 18 points or more.

First-line Therapies. The first-line therapy can be any therapy known in the art. For example, the first-line therapy can be a therapeutic agent that is, for example, a large molecule (biologic) or a small molecule, or an oral or parenteral inhibitor of intracellular signal transduction.

In some embodiments, the first-line therapy is therapeutic agent, and the agent is a small molecule inhibitor DMARD, such as methotrexate, and in other embodiments, the first-line therapy is a biologic, such as a TNF inhibitor, such as a TNF-α inhibitor, or an interleukin inhibitor, such as an IL-6, IL-17 or IL-12/IL-13 inhibitor. TNF-α inhibitors include, for example, the anti-TNF antibodies infliximab, adalimumab, certolizumab pegol, and golimumab, and the fusion protein etanercept. Etanercept (Enbrel®) is a fusion between soluble TNF receptor 2 and the Fc component of immunoglobulin G1. The anti-IL-6 antibody tocilizumab is an example of an IL-6 inhibitor. Other biologic therapeutics for treatment of arthritis include B cell-depleting agents, such as the anti-CD20 antibody rituximab (Rituxan, Genentech, Inc., South San Francisco, Calif.; and IDEC Pharmaceutical, San Diego, Calif.), and T cell costimulatory blocking agents, such as abatacept, which is a fusion protein composed of an immunoglobulin fused to the extracellular domain of CTLA-4.

In some embodiments, the first-line therapy is therapeutic agent, such as an inhibitor, such as a small molecule inhibitor, of a member(s) of the Janus kinase (JAK) family or Spleen tyrosine kinase (SYK) family. Members of these families are essential for the signaling pathways of various cytokines and are implicated in the pathogenesis of rheumatoid arthritis (RA), a representative autoimmune inflammatory disease. Members of the JAK family include JAK1, JAK2, JAK3, and Tyk2. An exemplary JAK inhibitor is the orally available JAK3 inhibitor CP-690,550 (tofacitinib). Members of the SYK family include SYK and chain-associated protein kinase (ZAP-70). Exemplary SYK inhibitors are R406, and its prodrug R788 (fostamatinib disodium).

The first-line therapy can also be an anti-Very Late Antigen-2 (VLA-2) antibody, such as GBR 500 (Sanofi, Bridgewater, N.J.), an anti-MAdCAM-1 antibody, such as vedolizumab, or an anti-CD20 antibody, such as rituximab.

In one embodiment, the patient has arthritis, and the first-line therapy includes treatment with a DMARD, a TNF-α inhibitor, a JAK (Janus Kinase) inhibitor, a SYK (Spleen Tyrosine Kinase) inhibitor, an IL-6 inhibitor, an IL-17 inhibitor, an IL-12/IL-23 inhibitor, a VLA-2 inhibitor, a CD20 inhibitor, or another biologic therapeutic. DMARDs include, for example, methotrexate, gold salts, leflunomide, sulfasalazine, or hydroxychloroquine.

In yet another embodiment, the patient has an inflammatory bowel disorder, such as Crohn's Disease or ulcerative colitis, and the first-line therapy includes treatment with an anti-MAdCAM-1 antibody, such as vedolizumab.

First-line therapies for treatment of arthritis also include, for example, hot and cold treatments, and splints or orthotic devices used to support and align joints. An arthritic patient may also undergo water therapy, ice massage, or transcutaneous nerve stimulation (TENS). Capsaicin cream can also be applied to the skin over the joints to provide pain relief, and the patient can take glucosamine and chondroitin. Patients can take acetaminophen for relief, or an NSAID (nonsteroidal anti-inflammatory drug), such as aspirin, ibuprofen or naproxen. Patients (particularly patients with autoimmune arthritis) can also receive corticosteroids, a COX-2 (cyclooxygenase-2) inhibitor, such as celecoxib, or an immunosuppressant, such as azathioprine or cyclophosphamide. A patient may also have surgery to rebuild a joint (arthroplasty) or to replace a joint. The patient may be on an exercise regimen, such as a low-impact aerobic activity to build or maintain endurance, range of motion exercises for flexibility, and strength training for muscle tone.

An “effective amount” of a therapy, for example, a first-line or second-line therapeutic agent, is delivered in an amount sufficient to cause beneficial or desired clinical results. An effective amount of a therapeutic agent can be delivered in one or more administrations. An “effective amount” of a first-line therapy will produce an “adequate response.” An “adequate response” is manifested as an improvement in symptoms, such as a decrease in swollen joint count and/or tender joint count, or a decrease in joint pain. An “effective amount” of an anti-VLA-1 antibody is an amount sufficient to palliate, ameliorate, stabilize, reverse, slow or delay progression of arthritis, or a symptom of arthritis, in accordance with clinically acceptable standards.

A subject can be monitored for improvements in arthritic symptoms upon treatment with a first- or second-line therapy. For example, a subject can be monitored by assaying an ACR (American College of Rheumatology) score. For example, a score of ACR20 indicates that there is at least a 20% reduction in the total number of tender and swollen joints and a reduction of 20% in three of the following five parameters: physician global assessment of disease, patient global assessment of disease, patient assessment of pain, C-reactive protein or erythrocyte sedimentation rate, and degree of disability in Health Assessment Questionnaire (HAQ) score. Typically, a score of ACR20 indicates that a patient has significant improvement of arthritic symptoms following administration of a therapeutic agent. A patient can exhibit more significant improvements with scores of ACR50 or ACR70, for example.

If a patient does not demonstrate a score of at least ACR20, for example, a score of at least ACR50 or ACR70, following administration of a therapy, then the patient can receive a negative assessment, or be determined to have an inadequate response to the therapy. In some embodiments, the patient's ACR score is monitored over the course of one or two weeks, or one or two months, or longer. In some embodiments, a patient will not meet a predetermined criterion that requires an ACR score of ACR20, ARC50, or ACR70 after treatment with a first-line therapy, and the patient will be selected for treatment with an anti-VLA-1 antibody.

The HAQ is a validated questionnaire, self-administered by the patient, that includes twenty items relating to function and four items relating to aids and devices. The questions include eight subscales: dressing and grooming, arising, hygiene, reach, eating, walking, grip, and activities. Items are scored from 0 (able to function without difficulty) to 3 (unable to function). The HAQ disease index is a weighted sum of the scale scores, with a higher score indicating poorer function. Decreases in the HAQ disease index exceeding −0.19 to −0.22 (for example, −0.2 or −0.21) are considered to be clinically important.

If a patient does not exhibit an improvement (an increase) in HAQ score by at least 0.19, for example, by at least 0.22 or more following administration of a therapy, then the patient can receive a negative assessment, or be determined to have an inadequate response to the therapy. In some embodiments, the patient is monitored for an improvement in HAQ over the course of one or two weeks, or one or two months, or longer. In some embodiments, a patient will not meet a predetermined criterion that requires an improvement in HAQ score of at least 0.19 or 0.22 or more, and the patient will be selected for treatment with an anti-VLA-1 antibody.

A patient can also be monitored for improvements in arthritic symptoms upon treatment with a first- or second-line therapy by assaying for an improvement in DAS (Disease Activity Score). DAS is a measure of the activity of rheumatoid arthritis that incorporates the following parameters: the total number of tender and swollen joints, ESR, and patient assessment of disease activity (Van der Heijde et al., “Development of disease activity score based on judgment in clinical practice by rheumatologists” J. Rheumatol. 20:579-81, 1993). If a patient does not exhibit an improvement in DAS, such as a decrease in DAS by at least 1.6, by at least 1.8, by at least 2.0, by at least 2.5, by least 3.0, by least 3.2, by at least 3.6, or more, following administration of a therapy, then the patient can receive a negative assessment, or be determined to have an inadequate response to the therapy. In some embodiments, the patient is monitored for an improvement in DAS over the course of one or two weeks, or one or two months, or longer. In some embodiments, a patient will not meet a predetermined criterion that requires an improvement in DAS (a decrease in DAS) by at least 1.6, by at least 2.0, by at least 2.2, by at least 2.8, by at least 3.2, by at least 3.6, or more, and the patient will be selected for treatment with an anti-VLA-1 antibody. Typically, a DAS score of 2.6 or less indicates remission of RA, and a DAS score of 3.2 or less indicates low disease activity. In one embodiment, patient will not meet a predetermined criterion that is a DAS of 2.6 or less, or a patient will not meet a predetermined criterion that is a DAS of 3.2 or less.

The DAS for 28-joint counts (DAS28-CRP measure) includes a composite of 4 variables: number of tender joints out of 28 joints, number of swollen joints out of 28 joints, CRP (in mg/L), and subject assessment of disease activity measure on a Visual Analogue Scale (VAS) of 100 millimeters (mm) DAS28-CRP values range from 0 to 9.31, with higher scores indicating more disease activity. Typically, a DAS28-CRP score of 2.6 or less indicates remission of RA, and a DAS28-CRP score or 3.2 or less indicates low disease activity. In one embodiment, patient will not meet a predetermined criterion that is a DAS of 2.6 or less, or a patient will not meet a predetermined criterion that is a DAS28 of 3.2 or less.

A patient can also be monitored for improvements in arthritic symptoms by a count of the total number of tender and swollen joints. If the total number of tender and swollen joints does not decrease by, for example, more than 1, 2, 3 or more following administration of a therapy, then the patient can receive a negative assessment, or be determined to have an inadequate response to the therapy. In some embodiments, the patient is monitored for a decrease in swollen or tender joint counts over the course of one or two weeks, or one or two months, or longer. In some embodiments, a patient will not meet a predetermined criterion that requires a decrease in swollen or tender joint count of 1, 2, 3 or more, and the patient will be selected for treatment with an anti-VLA-1 antibody. In some embodiments, a patient will not meet a predetermined criterion that requires a decrease in swollen or tender joint count of 15%, 20%, or 30% or more, and the patient will be selected for treatment with an anti-VLA-1 antibody.

A patient can also be monitored for improvements in arthritic symptoms by radiographic methods, such as MRI, ultrasound or X-ray. These methods provide images that can reveal the extent of synovitis, erosive changes, and edema. Failure to see a decrease in the extent of synovitis, a decrease in the rate of erosion in the joint, or a decrease in edema, such as over the course of one or two weeks or one or two months, or longer, for example, can indicate that the patient has an inadequate response to a therapy. In some embodiments, a patient will not meet a predetermined criterion that requires a decrease in the extent of synovitis, a decrease in the rate of erosion in the joint, or a decrease in “bone edema” or “osteitis” by 15%, 20%, 30% or more, and the patient will be selected for treatment with an anti-VLA-1 antibody.

A patient can also be monitored for improvements in arthritic symptoms upon treatment with a first or second-line therapy by assaying for the number of VLA-1⁺ cells, such as VLA-1⁺ T cells or monocytes, in blood or synovial fluid. If the number of VLA-1⁺ cells does not decrease by, for example, more than 15%, 20% or 30% or more following administration of a therapy, then the patient can receive a negative assessment, or be determined to have an inadequate response to the therapy. In some embodiments, the patient is monitored for a decrease in VLA-1⁺ cells over the course of one or two weeks, or one or two months, or longer. In some embodiments, a patient will not meet a predetermined criterion that requires a decrease in VLA-1⁺ cells of 15%, 20%, 30% or more, and the patient will be selected for treatment with an anti-VLA-1 antibody.

In some embodiments, a patient will not meet a predetermined criterion that requires an improvement in both tender and swollen joint counts of at least 15%, 20%, 30% or more, and an improvement of at least 15%, 20%, or 30% or more in three of the remaining five core measures: patient's assessment of pain (on the basis of a visual-analogue scale ranging from 1 to 100, with higher scores indicating more pain); levels of acute-phase reactants, such as CRP; HAQ score; and patient and physician global assessment. Patient and physician global assessments are evaluated on a scale of 0 to 100, with higher numbers indicating more severe disease.

In some embodiments, a patient is monitored for improvements in IBD symptoms upon treatment with a first or second-line therapy by determining the number of liquid or soft stools per day, such as over a 7 day period; determining the extent of abdominal pain or the size or presence of an abdominal mass; determining hematocrit levels; monitoring for a deviation from standard weight; or determining the presence and size of anal fissures, fistulae or abscesses. In one embodiment, the symptoms are assayed and applied to an activity scale, such as the CDAI. If the CDAI score does not decrease by at least 50, at least 60, at least 70, or at least 80 or more following administration of a first- or second-line therapy, then the patient can receive a negative assessment, or be determined to have an inadequate response to the therapy. In some embodiments, the patient's CDAI score is monitored over the course of one or two weeks, or one or two months, or longer. In some embodiments, a patient will not meet a predetermined criterion that requires a decrease in CDAI score of at least 50, at least 60, at least 70, or at least 80, and the patient will be selected for treatment with an anti-VLA-1 antibody.

In some embodiments, the patient's score on the IBDQ is monitored in response to treatment with a first- or second-line therapy. For example, if the IBDQ score fails to increase by at least 15 points, at least 16 points, at least 17 points, at least 18 points or more following administration of a first- or second-line therapy, then the patient can receive a negative assessment, or be determined to have an inadequate response to the therapy. In some embodiments, the patient's IBDQ score is monitored over the course of one or two weeks, or one or two months, or longer. In some embodiments, a patient will not meet a predetermined criterion that requires an increase in IBDQ score of at least 15 points, at least 16 points, at least 17 points, or at least 18 points or more, and the patient will be selected for treatment with an anti-VLA-1 antibody.

Information regarding a patient's response to a first-line therapy can be acquired directly or indirectly. For example, information regarding the patient's response can be assessed by a clinician or caregiver who directly examines the patient for symptom improvements following administration of a first-line therapy. Alternatively, the information can be acquired indirectly, such as from patient records obtained from the records of a hospital or clinic, or clinician or caregiver, or from a database, such as an on-line database.

“Acquire” or “acquiring” as the terms are used herein, refer to obtaining possession of a physical entity, or a value, such as a numerical value, by “directly acquiring” or “indirectly acquiring” the physical entity or value. “Directly acquiring” means performing a process (for example, examining the patient or a patient sample), to obtain the physical entity or value. “Indirectly acquiring” refers to receiving the physical entity or value from another party or source, such as from a third party laboratory that directly acquired the physical entity or value.

Directly acquiring a physical entity includes performing a process that includes a physical change in a physical substance, such as a starting material. Exemplary changes include making a physical entity from two or more starting materials, shearing or fragmenting a substance, separating or purifying a substance, combining two or more separate entities into a mixture, performing a chemical reaction that includes breaking or forming a covalent or non-covalent bond.

Directly acquiring a value includes performing a process that includes a physical change in a sample or another substance,such as by performing an analytical process which includes a physical change in a substance, for example, a sample, analyte, or reagent (sometimes referred to herein as “physical analysis”), performing an analytical method, such as a method which includes one or more of the following: separating or purifying a substance, such as an analyte, or a fragment or other derivative thereof, from another substance; combining an analyte, or fragment or other derivative thereof, with another substance, such as a buffer, solvent, or reactant; or changing the structure of an analyte, or a fragment or other derivative thereof, such as by breaking or forming a covalent or non-covalent bond, between a first and a second atom of the analyte; or by changing the structure of a reagent, or a fragment or other derivative thereof, such as by breaking or forming a covalent or non-covalent bond, between a first and a second atom of the reagent.

“Analyzing” a sample includes performing a process that involves a physical change in a sample or another substance, such as a starting material. Exemplary changes include making a physical entity from two or more starting materials, shearing or fragmenting a substance, separating or purifying a substance, combining two or more separate entities into a mixture, performing a chemical reaction that includes breaking or forming a covalent or non-covalent bond. Analyzing a sample can include performing an analytical process which includes a physical change in a substance, such as a sample, analyte, or reagent (sometimes referred to herein as “physical analysis”), performing an analytical method, for example a method that includes one or more of the following: separating or purifying a substance, for example, an analyte, or a fragment or other derivative thereof, from another substance; combining an analyte, or fragment or other derivative thereof, with another substance, such as a buffer, solvent, or reactant; or changing the structure of an analyte, or a fragment or other derivative thereof, such as by breaking or forming a covalent or non-covalent bond, between a first and a second atom of the analyte; or by changing the structure of a reagent, or a fragment or other derivative thereof, such as by breaking or forming a covalent or non-covalent bond between a first and a second atom of the reagent.

In one embodiment, determining whether a patient has improvements in arthritic symptoms, includes one or more of evaluating the patient, or analyzing a sample from the patient, requesting evaluation of the patient or analysis of the sample, requesting results from evaluation of the patient or analysis of the sample, or receiving the results from evaluation of the patient or analysis of the sample. Generally, analysis can include one or both of performing the underlying method (for example, assaying for the number of VLA-1⁺ cells or monocytes in a patient sample) or receiving data from another who has performed the underlying method.

Anti-VLA-1 antibodies. Antibodies to VLA-1, such as to the α subunit, β subunit, or both subunits of VLA-1, are suitable for use in the methods described herein. In one embodiment, the anti-VLA-1 antibody binds to the al subunit of VLA-1. Exemplary anti-VLA-1 antibodies are disclosed, for example, in U.S. Pat. No. 7,358,054, which is incorporated herein by reference in its entirety. Suitable antibodies for use in the methods described herein include: antibodies having one, two, or three light chain (LC) CDRs and one, two or three heavy chain (HC) CDRs, and in an embodiment all six CDRs, having the sequence of an antibody disclosed in U.S. Pat. No. 7,358,054; antibodies wherein each of the CDRs differs by no more than 1 or 2 amino acids from the CDRs of an antibody disclosed in U.S. Pat. No. 7,358,054 (variant amino acids, when used in this context, can be independently, or as a group, conservative on non-conservative changes).

In one embodiment, an anti-VLA-1 antibody useful for the methods described herein includes a LC variable region, a HC variable region, or both, from an antibody disclosed in U.S. Pat. No. 7,358,054; an antibody that binds an overlapping epitope with, or competes for binding with an antibody disclosed in U.S. Pat. No. 7,358,054; an antibody having a LC variable region, a HC variable region, or both, having least 90, 95, or 99% amino acid homology with the corresponding portions of an antibody disclosed in U.S. Pat. No. 7,358,054; an antibody having a LC variable region which differs by no more than 10, 5, or 1 amino acid residue, a HC variable region which differs by no more than 10, 5, or 1 amino acid residue, or both, from the corresponding portions of an antibody disclosed in U.S. Pat. No. 7,358,054.

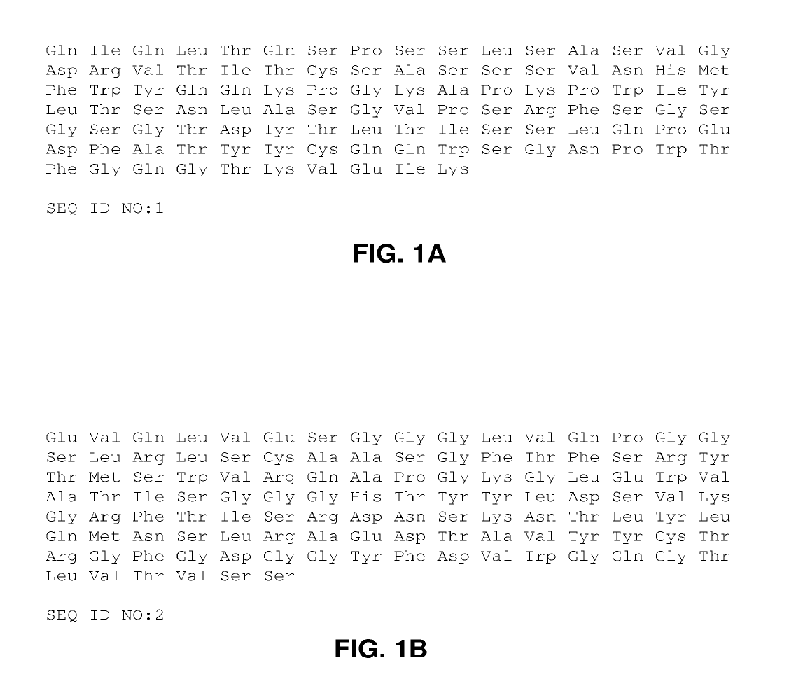

In one embodiment, an anti-VLA-1 antibody useful for the methods described herein includes a light chain variable region that is the same as or differs by no more than 10, 5, 3, or 1 amino acid from the sequence of SEQ ID NO:1 (FIG. 1A), and a heavy chain variable region that is the same as or differs by no more than 10, 5, 3, or 1 amino acid from the sequence of SEQ ID NO:2 (FIG. 1B).

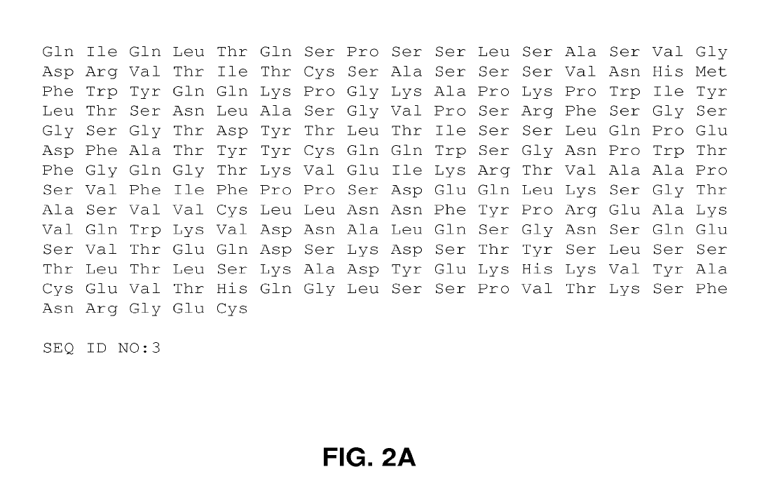

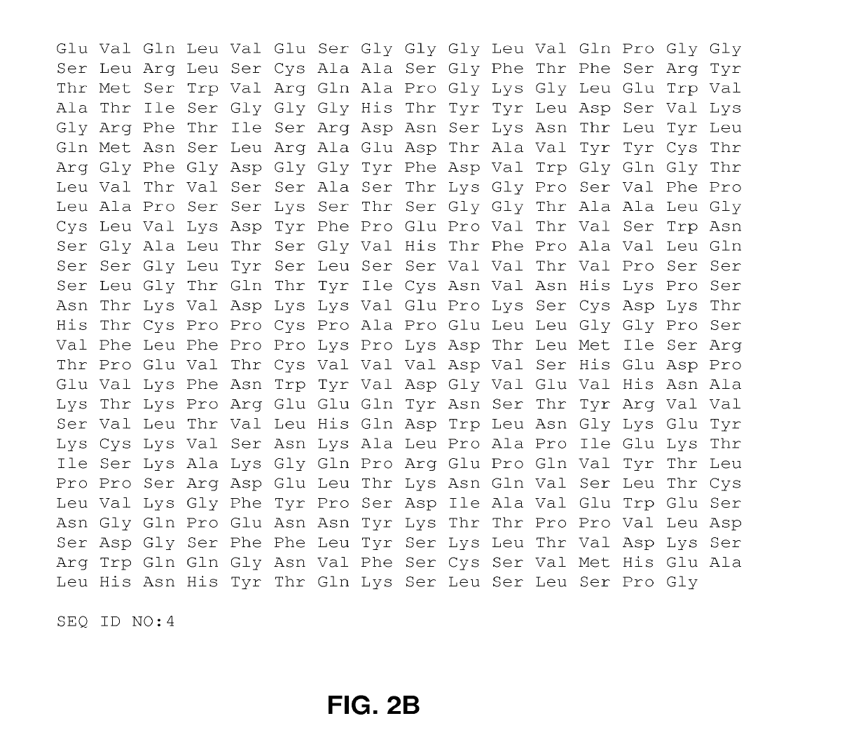

In one embodiment, an anti-VLA-1 antibody has a light chain sequence that is the same as or differs by no more than 10, 5, 3, or 1 amino acid from the sequence of SEQ ID NO:3 (FIG. 2A) and a heavy chain sequence that is the same as or differs by no more than 10, 5, 3, or 1 amino acid from the sequence of SEQ ID NO:4 (FIG. 2B).

As discussed herein, exemplary anti-VLA-1 antibodies useful in the methods described herein include the antibodies described in U.S. Pat. No. 7,358,054, which is incorporated herein by reference in its entirety. Antibodies described in U.S. Pat. No. 7,358,054, include, for example, monoclonal antibody AJH10 (ATCC PTA-3580; deposited on Aug. 2, 2001, with the American Type Culture Collection, 10801 University Boulevard, Manassas, Va. 20110-2209), hAQC2 (ATCC PTA-3275; deposited on Apr. 18, 2001), haAQC2 (ATCC PTA-3274; deposited on Apr. 18, 2001), hsAQC2 (ATCC PTA-3356; deposited on May 4, 2001) and mAQC2 (ATCC PTA-3273). All of these antibodies were deposited under the Budapest Treaty.

In one embodiment, an anti-VLA-1 antibody useful for the methods described herein includes a light chain polypeptide comprising the sequence of SEQ ID NO:1 (FIG. 1A), and a heavy chain polypeptide comprising the sequence of SEQ ID NO:2 (FIG. 1B).

In one embodiment, an anti-VLA-1 antibody has a light chain sequence comprising the sequence of SEQ ID NO:3 (FIG. 2A) and a heavy chain sequence comprising the sequence of SEQ ID NO:4 (FIG. 2B). Other anti-VLA-1 antibodies include, for example, monoclonal antibody 1B3 (ATCC IIB-10536) described in U.S. Pat. Nos. 5,391,481 and 5,788,966, and Ha31/8.

In one embodiment, an anti-VLA-1 antibody inhibits the interaction between VLA-1 and a VLA-1 ligand, such as collagen, by, for example, physically blocking the interaction, decreasing the affinity of VLA-1 for its counterpart, disrupting or destabilizing VLA-1 complexes, sequestering VLA-1, or targeting VLA-1 for degradation. In one embodiment, the antibody can bind to VLA-1 at one or more amino acid residues that participate in the VLA-1/ligand binding interface. Such amino acid residues can be identified by, for example, alanine scanning. In another embodiment, the antibody can bind to residues that do not participate in the VLA-1/ligand binding. For example, the antibody can alter a conformation of VLA-1 and thereby reduce binding affinity, or the antibody may sterically hinder VLA-1/ligand binding. In one embodiment, the antibody can reduce activation of a VLA-1-mediated event or activity.

Combination therapies. The anti-VLA antibodies for treatment of arthritis can be administered in place of, or in addition to, other therapies for arthritis.

In one embodiment, an anti-VLA-1 antibody is administered when a patient does not respond or stops responding to or improving in response to administration of, for example, a DMARD, a TNF-α inhibitor, a JAK (Janus Kinase) inhibitor (for example, a JAK1, JAK2 or JAK3 inhibitor), a SYK (Spleen Tyrosine Kinase) inhibitor, an IL-6 inhibitor, an IL-17 inhibitor, an IL-12/IL-23 inhibitor, a VLA-2 inhibitor, a MAd-CAM-1 inhibitor, a CD20 inhibitor, or another biologic antirheumatic therapy, such as abatacept. Exemplary DMARDs include methotrexate, leflunomide, sulfasalazine, hydroxychloroquine, gold salts, and penicillamine Exemplary TNF-α inhibitors include infliximab, adalimumab, certolizumab pegol, golimumab, and etanercept. An exemplary VLA-2 inhibitor is the anti-VLA-2 antibody GBR 500, an exemplary MAdCAM-1 inhibitor is the anti-MAd-CAM-1 antibody vedolizumab, and an exemplary CD20 inhibitor is the anti-CD20 antibody rituximab.

A patient can receive a DMARD, an anti-TNF-α therapy, or another therapeutic agent described herein as a first therapy, and then the patient can stop receiving the first therapy before receiving treatment with an anti-VLA-1 antibody. In one embodiment, the patient continues to receive the first therapeutic agent when the patient begins receiving the anti-VLA-1 therapy. For example, the patient receives an administration of the first therapeutic agent after an administration of the anti-VIA-1 antibody therapy, or the administrations are selected such that therapeutic levels of both the antibody and the first therapeutic agent are maintained in the patient. The antibody and the first therapeutic agent can be maintained in the patient for at least 1 day, at least 2 days, at least 5 days, at least 10 days or more.

In one embodiment, the patient receives an anti-TNF-α therapy and a DMARD therapy, then the patient stops receiving treatment with either or both of the anti-TNF-α and the DMARD therapy, and then the patient is administered an anti-VLA-1 antibody.

In one embodiment, a patient receives or continues to receive other treatments for arthritis while receiving treatment with an anti-VLA-1 antibody. For example, a patient may receive heat and cold treatments, or splints or orthotic devices can be employed to support and align joints. An arthritic patient may also undergo water therapy, ice massage, or transcutaneous nerve stimulation (TENS). Capsaicin cream can be applied to the skin over the joints to provide pain relief, and the patient can take glucosamine and chondroitin. Patients can also take acetaminophen, or an NSAID (nonsteroidal anti-inflammatory drug), such as aspirin, ibuprofen or naproxen. Patients (particularly patients with autoimmune arthritis) can also receive corticosteroids, a COX-2 (cyclooxygenase-2) inhibitor, such as celecoxib, or an immunosuppressant, such as azathioprine or cyclophosphamide. A patient may also have surgery to rebuild a joint (arthroplasty) or to replace a joint. The patient may be on an exercise regimen, such as a low-impact aerobic activity to build or maintain endurance, range of motion exercises for flexibility, and strength training for muscle tone.

Antibodies. As used herein, the term “antibody” refers to a protein that includes at least one immunoglobulin variable region, for example, an amino acid sequence that provides an immunoglobulin variable domain or an immunoglobulin variable domain sequence. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody” encompasses antigen-binding fragments of antibodies, including single chain antibodies, Fab fragments, F(ab′)2 fragments, Fd fragments, Fv fragments, and dAb fragments, as well as complete antibodies, such as intact and/or full length immunoglobulins of types IgA, IgG (for example, IgG1, IgG2, IgG3, IgG4), IgE, IgD, and IgM, and subtypes thereof. The light chains of the immunoglobulin may be of types kappa or lambda. In one embodiment, the antibody is glycosylated. An antibody can be functional for antibody-dependent cytotoxicity and/or complement-mediated cytotoxicity, or may be non-functional for one or both of these activities.

The VII and VL regions can be further subdivided into regions of hypervariability, termed “complementarity determining regions” (“CDR”), interspersed with regions that are more conserved, termed “framework regions” (FR). The extent of the FR's and CDRs has been precisely defined (see, Kabat, et al., Sequences of Proteins of Immunological Interest, Fifth Edition, U.S. Department of Health and Human Services, NIH Publication

No. 91-3242, 1991; and Chothia, et al., J. Mol. Biol. 196:901-917, 1987). Kabat definitions are used herein. Each VH and VL is typically composed of three CDR's and four FR' s, arranged from amino-terminus to carboxyl-terminus in the following order: FR1, CDR1, FR2, CDR2, FR3, CDR3, FR4. An “immunoglobulin domain” refers to a domain from the variable or constant domain of immunoglobulin molecules. Immunoglobulin domains typically contain two β-sheets formed of about seven β-strands, and a conserved disulphide bond (see, for example, Williams and Barclay, Ann. Rev Immunol. 6:381-405, 1988). An “immunoglobulin variable domain sequence” refers to an amino acid sequence that can form a structure sufficient to position CDR sequences in a conformation suitable for antigen binding. For example, the sequence may include all or part of the amino acid sequence of a naturally occurring variable domain. For example, the sequence may omit one, two or more N- or C-terminal amino acids, internal amino acids, may include one or more insertions or additional terminal amino acids, or may include other alterations. In one embodiment, a polypeptide that includes an immunoglobulin variable domain sequence can associate with another immunoglobulin variable domain sequence to form a target binding structure (or “antigen binding site”), such as a structure that interacts with VLA-1.

The VH or VL chain of the antibody can further include all or part of a heavy or light chain constant region, to thereby form a heavy or light immunoglobulin chain, respectively. In one embodiment, the antibody is a tetramer of two heavy immunoglobulin chains and two light immunoglobulin chains. The heavy and light immunoglobulin chains can be connected by disulfide bonds. The heavy chain constant region typically includes three constant domains, CH1, CH2, and CH3. The light chain constant region typically includes a CL domain. The variable region of the heavy and light chains contains a binding domain that interacts with an antigen. The constant regions of the antibodies typically mediate the binding of the antibody to host tissues or factors, including various cells of the immune system, such as effector cells,and the first component (CIq) of the classical complement system.

One or more regions of an antibody can be human, effectively human, or humanized. For example, one or more of the variable regions can be human or effectively human. For example, in a humanized antibody, typically, one or more of the CDRs, for example, HC CDR1, HC CDR2, HC CDR3, LC CDR1, LC CDR2, and LC CDR3, is non-human, such as rodent, for example, mouse, and other portions of the antibody are human. Typically, one or more of the framework regions can be human, for example, FR1, FR2, FR3, and FR4 of the HC or LC. In one embodiment, all the framework regions are human, such as derived from a human somatic cell, such as a hematopoietic cell that produces immunoglobulins, or a non-hematopoietic cell. In one embodiment, the human sequences are germline sequences, and thus are encoded by a germline nucleic acid. One or more of the constant regions can be human, effectively human, or humanized. In another embodiment, at least 70, 75, 80, 85, 90, 92, 95, or 98% of the framework regions (for example, FR1 , FR2, and FR3, collectively, or FR1, FR2, FR3, and FR4, collectively), or the entire antibody, can be human, effectively human, or humanized. For example, FR1, FR2, and FR3 collectively can be at least 70, 75, 80, 85, 90, 92, 95, 98, or 99% identical, or completely identical, to a human sequence encoded by a human germline segment. An “effectively human” immunoglobulin variable region is an immunoglobulin variable region that includes a sufficient number of human framework amino acid positions such that the immunoglobulin variable region does not elicit an immunogenic response in a normal human. An “effectively human” antibody is an antibody that includes a sufficient number of human amino acid positions such that the antibody does not elicit an immunogenic response in a normal human.

A “humanized” immunoglobulin variable region is an immunoglobulin variable region that is modified such that the modified form elicits less of an immune response in a human than does the non-modified form, for example, is modified to include a sufficient number of human framework amino acid positions such that the immunoglobulin variable region does not elicit an immunogenic response in a normal human. Descriptions of “humanized” immunoglobulins include, for example, U.S. Pat. Nos. 6,407,213 and 5,693,762. In some cases, humanized immunoglobulins can include a non-human amino acid at one or more framework amino acid positions. Anti-VLA-1 antibodies can also be chimeric antibodies, and thus generated by engineering a cognate antibody, such as a murine, rat or rabbit antibody. For example, a cognate antibody can be altered by recombinant DNA technology such that part or all of the hinge and/or constant regions of the heavy and/or light chains are replaced with the corresponding components of an antibody from another species, such as a human. Generally, the variable domains of the engineered antibody remain identical or substantially so to the variable domains of the cognate antibody. Such an engineered antibody is called a chimeric antibody and is less antigenic than the cognate antibody when administered to an individual of the species from which the hinge and/or constant region is derived. For example, a chimeric antibody having a human hinge and/or constant region, and framework regions from a mouse antibody, is less antigenic in a human than is the mouse antibody from which the FR regions were derived. Methods of making chimeric antibodies are well known in the art. Preferred constant regions include, but are not limited to, those derived from IgG1 and IgG4.

Antibody Generation. Antibodies that bind to VLA-1 can be generated by a variety of means, including immunization in an animal, and in vitro methods such as phage display. All or part of VLA-1 can be used as an immunogen or as a target for selection.

For example, VLA-1 or a fragment thereof, for example, all or a part of an al subunit of VLA-1, for example, an al-I domain, can be used as an immunogen. In one embodiment, the immunized animal contains immunoglobulin producing cells with natural, human, or partially human immunoglobulin loci. In one embodiment, the non-human animal includes at least a part of a human immunoglobulin gene. For example, it is possible to engineer mouse strains deficient in mouse antibody production with large fragments of the human Ig loci. Using the hybridoma technology, antigen-specific monoclonal antibodies derived from the genes with the desired specificity may be produced and selected. See, for example, XENOMOUSE™, Green et al., Nat. Gen. 7:13-21, 1994; U.S.2003-0070185; U.S. Pat. No. 5,789,650; and WO96/34096.

Non-human antibodies to VLA-1 can also be produced in a rodent. The non-human antibody can be humanized, such as by the methods described in EP 239 400 (Winter et al.); U.S. Pat. Nos. 6,602,503; 5,693,761; and 6,407,213. The non-human antibodies can alternatively be deimmunized, or otherwise modified to make them effectively human.

EP 239 400 describes altering antibodies by substitution (within a given variable region) of their complementarity determining regions (CDRs) for one species with those from another. Typically, CDRs of a non-human antibody, such as a mouse antibody, are substituted into the corresponding regions in a human antibody by using recombinant nucleic acid technology to produce sequences encoding the desired substituted antibody. Human constant region gene segments of the desired isotype (usually gamma I for CII and kappa for CL) can be added and the humanized heavy and light chain genes can be co-expressed in mammalian cells to produce a soluble humanized antibody. Other methods for humanizing antibodies can also be used. For example, other methods can account for the three dimensional structure of the antibody, framework positions that are in three dimensional proximity to binding determinants, and immunogenic peptide sequences. See, for example, WO 90/07861; U.S. Pat. Nos. 5,693,762; 5,693,761; 5,585,089; and 5,530,101; Tempest et al., Biotechnology 9:266-271, 1991, and U.S. Pat. No. 6,407,213.

At times, direct transfer of CDRs to a human framework leads to a loss of antigen-binding affinity of the resultant antibody. This is because in some cognate antibodies, certain amino acids within the framework regions interact with the CDRs and thus influence the overall antigen binding affinity of the antibody. In such cases, it would be critical to introduce “back mutations” in the framework regions of the acceptor antibody in order to retain the antigen-binding activity of the cognate antibody. The general approach of making back mutations is known in the art. For example, Queen et al., Proc. Natl. Acad. Sci. USA 86:10029-10033, 1989; Co et al, Proc. Nat. Acad. Sci. USA 88:2869-2873, 1991; and WO 90/07861 (Protein Design Labs Inc.) describe an approach that involves two key steps. First, the human V framework regions are chosen by computer analysis for optimal protein sequence homology to the V region framework of the cognate murine antibody. Then, the tertiary structure of the murine V region is modeled by computer in order to visualize framework amino acid residues that are likely to interact with the murine CDRs, and these murine amino acid residues are then superimposed on the homologous human framework. Under this two-step approach, there are several criteria for designing humanized antibodies. The first criterion is to use as the human acceptor the framework from a particular human immunoglobulin that is usually homologous to the non-human donor immunoglobulin, or to use a consensus framework from many human antibodies. The second criterion is to use the donor amino acid rather than the acceptor if the human acceptor residue is unusual and the donor residue is typical for human sequences at a specific residue of the framework. The third criterion is to use the donor framework amino acid residue rather than the acceptor at positions immediately adjacent to the CDRs.

One may also use a different approach, such as described in Tempest, Biotechnology 9:266-21, 1991. Under this approach, the V region frameworks derived from NEWM and REI heavy and light chains, respectively, are used for CDR-grafting without radical introduction of mouse residues. An advantage of using this approach is that the three-dimensional structures of NEWM and REI variable regions are known from X-ray crystallography and thus specific interactions between CDRs and V region framework residues can be readily modeled.

Fully human monoclonal antibodies that bind to VLA-1 can be produced, for example, using in vitro-primed human splenocytes, as described by Boerner et al., J. Immunol. 147:86-95, 1991. They may also be prepared by repertoire cloning as described by Persson et al., Proc. Nat. Acad. Sci. USA 88:2432-2436, 1991, or by Huang and Stollar, J. Immunol. Methods 141:227-236, 1991; also U.S. Pat. No. 5,798,230. Large nonimmunized human phage display libraries may also be used to isolate high affinity antibodies that can be developed as human therapeutics using standard phage technology (see, for example, Hoogenboom et al., Immunotechnology 4:1-20, 1998; Hoogenboom et al., Immunol Today 2:371-8, 2000; and U.S. 2003-0232333). Other methods for producing fully human antibodies involve the use of non-human animals that have inactivated endogenous Ig loci and are transgenic for un-rearranged human antibody heavy chain and light chain genes. Such transgenic animals can be immunized with cd-I domain or a desired antigenic fragment thereof, and hybridomas are then made from B cells derived therefrom. These methods are described in, for example, the various GenPharm/Medarex (Palo Alto, Calif.) publications/patents concerning transgenic mice containing human Ig mini-loci, such as U.S. Pat. No. 5,789, 650; the various Abgenix (Fremont, Calif.) publications/patents with respect to XENOMICE (for example, U.S. Pat. Nos. 6,075,181; 6,150,584 and 6,162,963; Green et al., Nature Genetics 7:13-21, 1994; and Mendez et al., Nat. Genet. 15:146-56, 1997); and the various Kirin (Japan) publications/patents concerning “transomic” mice (for example, EP 843 961, and Tomizuka et al., Nature Genetics 16:133-1443, 1997).

Antibodies described herein can be produced in prokaryotic and eukaryotic cells. In one embodiment, the antibodies (for example, scFv's) are expressed in a yeast cell such as Pichia (see, for example, Powers et al., J. Immunol. Methods 251:123-35, 2001), Hanseula, or Saccharomyces. Antibodies, particularly full length antibodies, such as full-length IgG antibodies, can be produced in mammalian cells. Exemplary mammalian host cells for recombinant expression include Chinese Hamster Ovary (CHO cells) (including dhfr⁻ CHO cells, described in Urlaub and Chasin, Proc. Natl. Acad. Sci. USA 77:4216-4220, 1980), used with a DHFR selectable marker, such as described in Kaufman and Sharp, Mol. Biol. 159:601-621 (1982); lymphocytic cell lines, such as NSO myeloma cells and SP2 cells, COS cells, K562, and a cell from a transgenic animal, such as a transgenic mammal. For example, the cell can be a mammary epithelial cell.

In addition to the nucleic acid sequence encoding the immunoglobulin domain, the recombinant expression vectors may carry additional nucleic acid sequences, such as sequences that regulate replication of the vector in host cells (for example, origins of replication) and selectable marker genes. The selectable marker gene facilitates selection of host cells into which the vector has been introduced (see, for example, U.S. Pat. Nos. 4,399,216; 4,634,665; and 5,179,017). Exemplary selectable marker genes include the dihydrofolate reductase (DHFR) gene, such asfor use in dhfr host cells with methotrexate selection/amplification, and the neo gene, such as for G418 selection.

In an exemplary system for recombinant expression of an antibody, such as a full length antibody or an antigen-binding portion thereof, a recombinant expression vector encoding both the antibody heavy chain and the antibody light chain is introduced into dhfr⁻ CHO cells by calcium phosphate-mediated transfection. Within the recombinant expression vector, the antibody heavy and light chain genes are each operatively linked to enhancer/promoter regulatory elements, such as those derived from SV40, CMV, adenovirus and the like, such as a CMV enhancer/AdMLP promoter regulatory element or an SV40 enhancer/AdMLP promoter regulatory element, to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, to transfect the host cells, to select for transformants, to culture the host cells, and to recover the antibody from the culture medium. For example, some antibodies can be isolated by affinity chromatography with a Protein A or Protein G.

Antibodies may also include modifications, such as modifications that alter Fc function, such as to decrease or remove interaction with an Fc receptor or with CIq, or both. For example, the human IgG1 constant region can be mutated at one or more residues, for example, one or more of residues 234 and 237 (according to the numbering in U.S. Pat. No. 5,648,260). Other exemplary modifications include those described in U.S. Pat. No. 5,648,260.

For some antibodies that include an Fc domain, the antibody production system may be designed to synthesize antibodies or other proteins in which the Fc region is glycosylated. For example, the Fc domain of IgG molecules is glycosylated at asparagine 297 in the CH2 domain. The Fc domain can also include other eukaryotic post-translational modifications. In other cases, the protein is produced in a form that is not glycosylated. Antibodies can also be produced by a transgenic animal. For example, U.S. Pat. No. 5,849,992 describes a method for expressing an antibody in the mammary gland of a transgenic mammal. A transgene is constructed that includes a milk-specific promoter and nucleic acid sequences encoding the antibody of interest, such as an antibody described herein, and a signal sequence for secretion. The milk produced by females of such transgenic mammals includes, secreted-therein, the protein of interest, for example, an antibody. The protein can be purified from the milk, or for some applications, used directly.

An anti-VLA-1 antibody may further include other moieties to effect the desired functions. For example, the antibody may include a toxin moiety, such as tetanus toxoid or ricin, or a radionuclide, such as ¹¹¹In or ⁹⁰Y, such as for killing cells targeted by the antibodies (see, for example, U.S. Pat. No. 6,307,026). The antibodies may include a moiety, such as a biotin, fluorescent moiety, a radioactive moiety, a histidine tag, etc., for easy isolation or detection. The antibodies may also include a moiety that can prolong their serum half life, for example, a polyethylene glycol (PEG) moiety.

Pharmaceutical compositions. An anti-VLA-1 antibody can be formulated as a pharmaceutical composition, such as for administration to a subject to treat arthritis, for example, rheumatoid arthritis. Typically, a pharmaceutical composition includes a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. The composition can include a pharmaceutically acceptable salt, such as an acid addition salt or a base addition salt (see, for example, Berge, et al., J. Pharm. Sci. 66:1-19, 1977). The VLA-1 antagonist can be formulated according to standard methods. Pharmaceutical formulation is a well-established art, and is further described, for example, in Gennaro (ed.), Remington: The Science and Practice of Pharmacy, 20^{th} ed., Lippincott, Williams & Wilkins (2000) (ISBN: 0683306472); Ansel et al., Pharmaceutical Dosage Forms and Drug Delivery Systems, 7^{th} Ed., Lippincott Williams & Wilkins Publishers (1999) (ISBN: 0683305727); and Kibbe (ed.), Handbook of Pharmaceutical Excipients American Pharmaceutical Association, 3^{rd} ed. (2000) (ISBN: 091733096X).

In one embodiment, an anti-VLA-1 antibody can be formulated with excipient materials, such as sodium chloride, sodium dibasic phosphate heptahydrate, sodium monobasic phosphate, and a stabilizer. The antibody can be provided, for example, in a buffered solution at a suitable concentration and can be stored at 2 ° C. to 8 ° C. The pharmaceutical compositions may be in a variety of forms. These include, for example, liquid, semi-solid and solid dosage forms, such as liquid solutions that are, for example, injectable or infusible; dispersions or suspensions; tablets; pills; powders; liposomes and suppositories. The preferred form can depend on the intended mode of administration and therapeutic application. Typically, compositions for the agents described herein are in the form of injectable or infusible solutions.

Such anti-VLA-1 antibody compositions can described herein can be administered orally or parenterally, such as by intravenous, subcutaneous, intraperitoneal, or intramuscular injection.

The phrases “parenteral administration” and “administered parenterally” as used herein, mean modes of administration other than enteral and topical administration (usually by injection), and include, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural, intracerebral, intracranial, intracarotid and intrasternal injection and infusion.

The composition can be formulated as a solution, microemulsion, dispersion, liposome, or other ordered structure suitable for stable storage at high concentration. Sterile injectable solutions can be prepared by incorporating an agent described herein in the required amount in an appropriate solvent with one or a combination of ingredients enumerated above, as required, followed by filtered sterilization. Generally, dispersions are prepared by incorporating an agent described herein into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of an agent described herein plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

In certain embodiments, the VLA-1 antagonist may be prepared with a carrier that will protect the compound against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known. See, for example, J. R. Robinson, ed., Sustained and Controlled Release Drug Delivery Systems, Marcel Dekker, Inc., New York, 1978.

An anti-VLA-1 antibody can be modified, such as with a moiety that improves its stabilization and/or retention in circulation, such as in blood, serum, or other tissues, for example, by at least 1.5-fold, at least 2-fold, at least 5-fold, at least 10-fold, or at least 50-fold. The modified antibody can be evaluated to assess whether it can reach sites of damage, such as an arthritic joint, for example, by using a labeled form of the anttibody.

For example, the anti-VLA-1 antibody can be associated with a polymer, for example, a substantially non-antigenic polymer, such as a polyalkylene oxide or a polyethylene oxide. Suitable polymers will vary substantially by weight. Polymers having molecular number average weights ranging from 200 to 35,000 Daltons (or ranging from about 1,000 Daltons to 15,000 Daltons, or ranging from about 2,000 Daltons to 12,500 Daltons) can be used.

In one embodiment, an anti-VLA-1 antibody can be conjugated to a water soluble polymer, such as a hydrophilic polyvinyl polymer, such as polyvinylalcohol or polyvinylpyrrolidone. A non-limiting list of such polymers include polyalkylene oxide homopolymers, such as polyethylene glycol (PEG) or polypropylene glycols, polyoxyethylenated polyols, copolymers thereof and block copolymers thereof, provided that the water solubility of the block copolymers is maintained. Additional useful polymers include polyoxyalkylenes, such as polyoxyethylene, polyoxypropylene, and block copolymers of polyoxy ethylene and polyoxypropylene, for example, Pluronic; polymethacrylates; carbomers; and branched or unbranched polysaccharides.

When the anti-VLA-1 antibody is used in combination with a second agent, such as an anti-inflammatory agent, or a DMARD, the two agents can be formulated separately or together. For example, the respective pharmaceutical compositions can be mixed, such as just prior to administration, and administered together or can be administered separately. The respective pharmaceutical composition can be administered separately, and at the same or at different times.

Administration. An anti-VLA-1 antibody featured in the invention can be administered to a subject, such as a human subject, by a variety of methods. For many applications, the route of administration is one of: intravenous injection or infusion (IV), subcutaneous injection (SC), intraperitoneal administration (IP), or intramuscular injection.

In some cases, administration may be directly into the CNS, such as by intrathecal, intracerebroventricular (ICV), intracerebral or intracranial administration. The antagonist can be administered as a fixed dose, or in, for example, a mg/kg dose.

The dose can also be chosen to reduce or avoid production of antibodies against the antagonist.

The route and/or mode of administration of the blocking agent can also be tailored for the individual case. Dosage regimens arc adjusted to provide the desired response, for example, a therapeutic response or a combinatorial therapeutic effect.

Generally, any combination of doses (either separate or co-formulated) of the anti-VLA-1 antibody (and optionally a second agent) can be used in order to provide a subject with the agent in bioavailable quantities. For example, doses in the range of 0.025 mg/kg to 100 mg/kg, 0.05 mg/kg to 50 mg/kg, 0.1 mg/kg to 30 mg/kg, 0.1 mg/kg to 5 mg/kg, or 0.3 mg/kg to 3 mg/kg can be administered. Other suitable dosage levels include, for example, between 0.001 mg/kg and 100 mg/kg body weight per administration, between 0.1 mg/kg and 50 mg/kg body weight per administration, between 0.1 mg/kg body weight and 20 mg/kg body weight, such as between 0.1 mg/kg body weight and 10 mg/kg body weight per administration. In other embodiments, the antibody is administered at a dose of 0.3 mg/kg to 1 mg/kg, or 5 to 12.5 mg/kg per administration.

In another aspect, the invention features a method of treating a subject for arthritis, comprising administering to the subject an anti-VLA-1 antibody, such as an anti-VLA-1 antibody described herein, according to a regimen selected from the following: 0.1 mg/kg to 1 mg/kg; 0.2 mg/kg to 1 mg/kg; 0.3 mg/kg to 1 mg/kg; 0.4 mg/kg to 1 mg/kg; 0.2 mg/kg to 4 mg/kg; and 0.3 mg/kg to 5 mg/kg.

In another aspect, the invention features a method of treating a subject for arthritis, comprising administering to the subject an anti-VLA-1 antibody, for example, an anti-VLA-1 antibody described herein, according to a regimen selected from the following: 5 mg/kg to 10 mg/kg; 6 mg/kg to 9 mg/kg; 7 mg/kg to 8 mg/kg; 5 mg/kg to 9 mg/kg; 5 mg/kg to 8 mg/kg; 5 mg/kg to 7 mg/kg; 6 mg/kg to 10 mg/kg; 7 mg/kg to 10 mg/kg and 8 mg/kg to 10 mg/kg.

In another aspect, the invention features a method of treating a subject for arthritis, comprising, administering to the subject, an anti-VLA-1 antibody, for example, an anti-VLA-1 antibody described herein, according to a regimen selected from the following: 0.03 mg/kg to less than 0.1 mg/kg; 0.03 mg/kg to 0.9 mg/kg; 0.03 mg/kg to 0.08 mg/kg;

0.03 mg/kg to 0.05 mg/kg; 0.04 mg/kg to 0.08 mg/kg; 0.04 mg/kg to 0.07 mg/kg; or 0.05 mg/kg to less than 0.1 mg/kg.

In another aspect, the invention features a method of treating a subject for arthritis, comprising administering to the subject an anti-VLA-1 antibody, for example, an anti-VLA-1 A-1 antibody described herein, according to a regimen selected from the following: greater than 1.0 mg/kg to less than 5.0 mg/kg; greater than 1.0 mg/kg to 2.0 mg/kg; greater than 1.0 mg/kg to 3.0 mg/kg; greater than 1.0 mg/kg to 4.0 mg/kg; 2.0 mg/kg to less than 5 mg/kg; 3.0 mg/kg to less than 5 mg/kg; 4.0 mg/kg to less 5 mg/kg; 2 mg/kg to 3 mg/kg; 3 mg/kg to 4 mg/kg; 1.5 mg/kg to 2.5 mg/kg; 2.5 mg/kg to 3.5 mg/kg; or 3.5 mg/kg/day to 4.5 mg/kg.

In another aspect, the invention features a method of treating a subject for arthritis, comprising administering to the subject an anti-VLA-1 antibody, for example, an anti-VLA-1 antibody described herein, according to a regimen selected from the following: 0.03 mg/kg per administration; 0.1 mg/kg per administration; 0.2 mg/kg per administration; 0.3 mg/kg per administration; 0.5 mg/kg per administration; 0.6 mg/kg per administration; 0.8 mg/kg per administration; 1 mg/kg per administration; 3 mg/kg per administration; 5 mg/kg per administration; 7 mg/kg per administration; 8 mg/kg per administration; 10 mg/kg per administration, and 12.5 mg/kg per administration.

In certain embodiments, a composition having an anti-VLA-1 antibody is administered in an amount effective to provide a plasma level of antibody of at least 1 μg/ml. The dose can be, for example, per administration or per day.

In some embodiments, an anti-VLA-1 antibody is administered once every 3 to 10 days, for example, once every 3 days, 4 days, 5 days, or 6 days; once every 8 to 16 days;

or once every 12 to 30 days. In some embodiments, an anti-VLA-1 antibody is administered every 40 days, every 45 days, every 50 days, every 55 days, every 60, every 70 days, every 80 days, every 90 days, every 100 days or every 120 days.

In some embodiments, the patient receives at least 2, at least 3, at least 4, at least 5, or at least 6 administrations before a drug holiday or cessation.

Administration can be in a single administration, or administration can be in intervals, such as part of a treatment regimen. For example, an anti-VLA-1 antibody can be administered once or twice or three times per day, once or twice or three times per week, once every two or three or four weeks, or once or twice or three times per month. In one embodiment the antibody is administered every 1 to 14 days.

In some embodiments, an anti-VLA-1 antibody is administered subcutaneously or intramuscularly or intravenously once or twice per week, or once or twice per month. In one embodiment, the anti-VLA-1 antibody is administered subcutaneously twice per week.

In some embodiments, a loading dose is provided initially, which is followed by a series of maintenance doses. The antibody concentration and route of administration for the loading dose can be the same as, or different than, the antibody concentration and route of administration of the maintenance doses. For example, a loading dose can be administered intravenously and maintenance doses can be provided subcutaneously.

Dosage unit form or “fixed dose” as used herein refers to physically discrete units suited as unitary dosages for the subjects to be treated. Each unit contains a predetermined quantity of active compound calculated to produce the desired therapeutic effect in association with the required pharmaceutical carrier and optionally in association with the other agent.

Single or multiple dosages may be given to an arthritic patient. In one embodiment, the anti-VLA-1 antibody may be administered by continuous infusion. The treatment can continue for days, weeks, months or years to manage the symptoms of arthritis, or to prevent progression of the disease.

A pharmaceutical composition may include a therapeutically effective amount of an anti-VLA-1 antibody. Such effective amounts can be determined based on the effect of the administered antibody, or the combinatorial effect of the antibody and secondary agent if a secondary agent is used. A therapeutically effective amount of an antibody may also vary according to factors such as the disease state, age, sex, and weight of the individual, and the ability of the compound to elicit a desired response in the individual, for example, the improvement of at least one disorder parameter, such as a decrease in pain or in the swelling of an affected joint. A therapeutically effective amount is also one in which any toxic or detrimental effects of the composition are outweighed by the therapeutically beneficial effects.

Kits. An anti-VLA-1 antibody can be provided in a kit. For example, the kit can include (a) a container that contains a composition that includes an anti-VLA-1 antibody, and optionally (b) informational material. The informational material can be descriptive, instructional, marketing or other material that relates to the methods described herein and/or the use of the antibodies for therapeutic benefit. The kit can optionally include a second agent, such as a DMARD or a TNF-α inhibitor, for treating arthritis. For example, the kit includes a first container that contains a composition that includes the anti-VLA-1 antibody and a second container that includes the second agent.

In addition to the antibody, a composition in the kit can include other ingredients, such as solvent or buffer, a stabilizer, or a preservative. The anti-VLA-1 antibody can be provided in any form, such as in liquid, dried or lyophilized form, and the formulation is typically substantially pure and/or sterile. When the agents are provided in a liquid solution, the liquid solution is typically an aqueous solution. When the agents are provided as a dried form, reconstitution generally is by the addition of a suitable solvent. The solvent, for example, sterile water or buffer, can optionally be provided in the kit.

The kit can include one or more containers for the composition or compositions containing the agents. For example, the kit can contain separate containers, dividers or compartments for the composition(s) and informational material. In one example, the antibody composition can be contained in a bottle, vial, or syringe, and the informational material can be contained in a plastic sleeve or packet. In some embodiments, the separate elements of the kit are contained within a single, undivided container. For example, the composition is contained in a bottle, vial or syringe that has attached thereto the informational material in the form of a label. In some embodiments, the kit includes a plurality, for example, a pack, of individual containers, each containing one or more unit dosage forms, for example, one or more dosage forms described herein, of the anti-VLA-1 antibody. The containers optionally include a combination unit dosage, for example, a unit dosage form that includes both the anti-VLA-1 antibody and the second agent, such as in a desired ratio. For example, the kit can include a plurality of syringes, ampoules, foil packets, blister packs, or medical devices, each containing a single combination unit dose. The containers of the kits can be air tight; waterproof, for example, impermeable to changes in moisture or evaporation; and/or light-tight.

The informational material provided in a kit can include information about production of the antibody, molecular weight of the antibody, concentration, date of expiration, batch or production site information, and so forth. The informational material may also relate to methods of administering the anti-VLA-1 antibody, such as in a suitable dose, dosage form, or mode of administration, such as a dose, dosage form, or mode of administration described herein, to treat a subject who has arthritis. The information can include information about who should or should not receive the anti-VLA-1 antibody as a therapy for arthritis. For example, the informational material may specify that a patient not receive a DMARD or an anti-TNF-α therapy for a certain amount of time, for example, 3 weeks, 4 weeks, 5 weeks, one month, or more, prior to starting treatment with an anti-VLA-1 antibody therapy.

The informational material of the kits is not limited in its form. The informational material can be provided in a variety of formats, including printed text, drawings, or photographs, such as on a label or printed sheet. Other suitable formats include computer readable material, video recording, or audio recording. The informational material can include contact information, such as a physical address, email address, website, or telephone number, where a user of the kit can obtain substantive information about the anti-VLA-1 antibody and/or its use in the methods described herein.

The kit optionally includes a device suitable for administration of the composition, for example, a syringe or other suitable delivery device. The device can be provided pre-loaded with one or more therapeutic agents, or can be empty, but suitable for loading.

Other embodiments are in the claims.

What is claimed is:

1. A method of treating a patient with an anti-VLA-1 antibody, wherein said patient was previously administered a first therapeutic agent, and wherein said patient's response to the first therapeutic agent was inadequate, comprising:
administering an effective amount of an anti-VLA-1 antibody to said patient, thereby treating said patient.

2. The method of claim 1, wherein the response wass determined to be inadequate because (i) the patient failed to have an improvement in arthritic symptoms; (ii) the patient ceased to have improvement in arthritic symptoms; or (iii) the patient experienced a worsening of arthritic symptoms.

3. The method of claim 2, wherein improvement comprises a decrease in swollen joint count or tender joint count.

4. The method of claim 2, wherein a worsening of arthritic symptoms comprises an increase in swollen joint count or tender joint count.

5. The method of claim 1, wherein the patient has arthritis.

6. The method of claim 1, wherein the first therapeutic agent is a DMARD.

7. The method of claim 5, wherein the DMARD is methotrexate, leflunomide, sulfasalazine, hydroxychloroquine, or gold salts.

8. The method of claim 1, wherein the patient was diagnosed with rheumatoid arthritis for at least 6 months.

9. The method of claim 1, wherein administration of the first therapeutic agent was stopped before the patient is administered the anti-VLA-1 antibody.

10. The method of claim 1, wherein administration of the first therapeutic agent was stopped for at least 4 weeks before the patient is administered the anti-VLA-1 antibody.

11. The method of claim 1, wherein administration of the first therapeutic agent is continued while the patient is administered the anti-VLA-1 antibody.

12. The method of claim 11, wherein the first therapeutic agent is a DMARD, and the DMARD is methotrexate, administered at a dose of 25 mg/week or less; leflunomide, administered at a dose of 20 mg/day or less; sulfasalazine, administered at a dose of 3000 mg/day or less; or hydroxychloroquine, administered at a dose of 400 mg/day or less.

13. The method of claim 11, wherein the first therapeutic agent is a DMARD, and the patient is not administered more than one DMARD therapy while the patient is administered the anti-VLA-1 antibody.

14. The method of claim 11, wherein the first therapeutic agent is hydroxychloroquine, and the patient is further administered a second DMARD while the patient is administered the anti-VLA-1 antibody.

15. The method of claim 1, wherein the first therapeutic agent is a TNF-α inhibitor

16. The method of claim 1, wherein the anti-VLA-1 antibody comprises a light chain comprising the sequence of SEQ ID NO:1, and a heavy chain comprising the sequence of SEQ ID NO:2.

17. The method of claim 1, wherein the anti-VLA-1 antibody comprises a light chain comprising the sequence of SEQ ID NO:3, and a heavy chain comprising the sequence of SEQ ID NO:4.

18. The method of claim 1, wherein the anti-VLA-1 antibody binds the same epitope as an antibody comprising a light chain comprising the sequence of SEQ ID NO:1, and a heavy chain comprising the sequence of SEQ ID NO:2.

19. A method of selecting a patient as a candidate to receive treatment with an anti-VLA-1 antibody, wherein the patient previously has been administered a first therapeutic agent, the method comprising:
a) performing a test on a patient sample to assess a patient's response to the first therapeutic agent; and
b) if said patient response to the first therapeutic agent fails to meet a predetermined criterion, selecting the patient as a candidate for treatment with an anti-VLA-1 antibody, and if said response does meet that predetermined criterion, determining that the patient is not a candidate to receive treatment with the anti-VLA-1 antibody.

20. A method of selecting or classifying a patient as a candidate to receive treatment with an anti-VLA-1 antibody, wherein the patient previously has been administered a first therapeutic agent, the method comprising:
a) assessing a patient's response to said first therapeutic agent, wherein said assessing comprises analyzing a sample from said patient; and
b) if said response fails to meet a predetermined criterion, selecting or classifying the patient as a candidate for treatment with an anti-VLA-1 antibody, and if said response meets a predetermined criterion, selecting or classifying the patient as not a candidate to receive treatment with the anti-VLA-1 antibody,
thereby selecting or classifying said patient as a candidate to receive treatment with an anti-VLA-1 antibody.
